(19) United States
(12) Patent Application Publication
(10) Pub. No.: US 2008/0097561 A1
(43) Pub. Date: Apr. 24, 2008

(54) DUAL CYCLE THERMAL SYSTEM AND METHOD OF USE

(75) Inventors: Gerald Melsky, Lexington, MA (US); Charles D. Lennox, Hudson, NH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: MedCool, Inc., Wellesley, MA (US)

(21) Appl. No.: 11/870,447

(22) Filed: Oct. 11, 2007

Related U.S. Application Data
(60) Provisional application No. 60/852,599, filed on Oct. 18, 2006.

(51) Int. Cl. A61B 18/00

(52) U.S. Cl. 607/104

(57) ABSTRACT

Various types of body heating or cooling devices are described in the present disclosure. Such devices, and associated methods, can utilize two separated thermal transfer fluids to promote heating or cooling of a body part. For example, a cooling cap can include a rigid shell and a flexible seal that form a fluid containment space with a patient's head. The rigid shell can include a fluid circulation apparatus disposed within the fluid containment space, and be coupled to a console pump to form a closed loop circulation system. A volume of fluid can be introduced into the fluid containment space to contact the patient's scalp. The console pump can then circulate a thermal transfer fluid through the fluid circulation apparatus to reduce the temperature of a fluid held within the fluid containment space.

### CROSS REFERENCE TO RELATED APPLICATIONS

The present application claims the benefit of a U.S. Provisional Patent Application bearing Ser. No. 60/852,599, filed Oct. 18, 2006, entitled “Dual Cycle Cooling System and Method of Use.” The entire contents of the provisional patent application are hereby incorporated by reference herein.

The present application is related to a copending PCT International Patent Application, bearing International Application No. PCT/US2006/012561 and having International Filing Date Apr. 3, 2006; which claims the benefit of U.S. Provisional Patent Application Ser. No. 60/669,336, filed Apr. 7, 2005. The present application is also related to copending U.S. patent application Ser. No. 11/243,237, filed Oct. 4, 2005, which is a continuation of U.S. Pat. No. 6,962,600 issued Nov. 8, 2005. All these applications and the patent are hereby incorporated herein by reference in their entirety.

### FIELD OF THE INVENTION

The present invention generally relates to methods and devices for heat transfer with a patient, and more particularly to a methods and devices for heating or cooling a tissue region of interest.

### BACKGROUND OF THE INVENTION

Patients that suffer from stroke, cardiac arrest, or head trauma, as well as patients that have undergone invasive brain or vascular surgery, are at risk for ischemic injury, which can occur when an organ does not receive a sufficient supply of oxygen. For example, in the case where a patient suffers from a stroke, a clot blocks the blood supply to a portion of the patient's brain. As a result, the patient can experience a critical rise in intra-cranial pressure, brain cell death, and a loss of brain function.

To help minimize ischemic injury after such a traumatic event, systemic hypothermia can be induced in the patient. The effectiveness of systemic hypothermia therapy is a function of several factors including, for example, the level of cooling of the patient (between temperatures of approximately 30° C. and 35° C.), the amount of time that elapses between an original insult, such as cardiac arrest or heart attack, and achievement of protective levels of hypothermia, and the duration of the hypothermic state.

Systemic hypothermia has historically been applied to a patient by immersion of the patient's body in a cool bath where the depth and duration of hypothermia is limited by the patient's ability to tolerate the therapy. Currently, there are several conventional systemic hypothermia systems available. Such conventional systems include pads having fluid circulation channels disposed within the inner walls of the pads. The pads can be applied to a patient's body and cooled water can be circulated through the pads to cause a thermal exchange between the patient and the pad to induce systemic hypothermia in the patient.

Attempts have also been made to induce hypothermia in a patient by local cooling the surface of the patient's head. For example, certain head-cooling devices include a head cap with a gel-filled liner. Prior to use, the head cap is placed into a freezer to reduce the temperature of the gel. During use, the cap can be placed on the head of a patient such that thermal exchange occurs between the chilled liner and the patient's head to locally induce hypothermia in the head of the patient. However, the presence of hair and/or air pockets between the scalp of the patient and the liner walls can act as a thermal insulator and can minimize the effectiveness of the heat transfer between the patient's scalp and the cap.

There is a need for improved hypothermia devices that provide direct contact between a cooling fluid and a patient's scalp to induce local hypothermia within a patient.

### SUMMARY OF THE INVENTION

One exemplary embodiment is directed to a device for heating or cooling a patient's head. The device includes a head-covering, which can optionally comprise a shell, which can be adapted to surround at least a portion of the patient's head. When the head-covering is worn, the covering can define a fluid containment space (e.g., between the head-covering and the head of a patient) into which a first thermal transfer fluid can be introduced. The head covering can include a vent, which can comprise an air inlet and an air outlet. The inlet and outlet can be disposed at a caudal portion and a cephalic portion of the head covering, respectively. At least one fluid port can be configured in communication with the fluid containment space for introducing or removing fluid from the containment space. The device can also include a fluid circulation apparatus, which can optionally comprise a tubular structure, which is disposed at least partially with in the fluid containment space. The fluid circulation apparatus can be adapted to allow circulation of a second thermal transfer fluid through the fluid circulation apparatus while maintaining separation between the fluids. In particular, the fluid circulation apparatus can be adapted to promote heat transfer between the two fluids.

Another exemplary embodiment is directed to a method of heating or cooling a patient's head (e.g., to induce localized hypothermia in a patient). The method includes contacting the patient's head with a first thermal transfer fluid. A cap can be utilized to contain the first thermal transfer fluid between the cap and the patient's head. In one instance, the cap can be placed on the patient's head, followed by introducing the first thermal fluid into the cap. Heat can be transferred between the first thermal transfer fluid and a second thermal transfer fluid, the fluids being separated. The second thermal transfer fluid can be thermally regulated to control heating or cooling of the patient's head. For example, the second thermal transfer fluid can be circulated in a closed loop circulation apparatus to provide the thermal regulation.

### BRIEF DESCRIPTION OF THE DRAWINGS

The invention will be more fully understood from the following detailed description taken in conjunction with the accompanying drawings, in which:

FIG. 1 is a schematic view of an embodiment of a thermal regulation system that includes a head covering device;

FIG. 2 is a side sectional view of an embodiment of the head covering device shown in FIG. 1; and

FIG. 3 illustrates a resuscitation system that includes the thermal regulation system, such as shown in FIG. 1.

### DETAILED DESCRIPTION

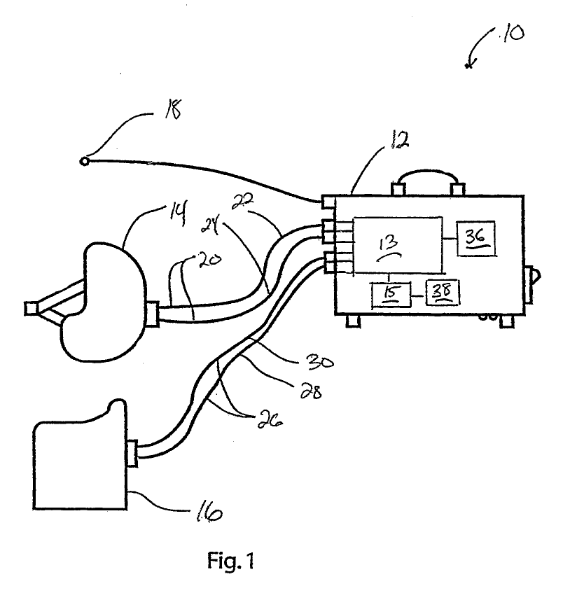

FIG. 1 illustrates an embodiment of a thermal regulation system 10 that is generally operable to induce localized hypothermia in a patient. The thermal regulation system 10 includes a console 12 that includes a reservoir 13 containing a thermal regulation fluid, such as cooling fluid. Though some embodiments discussed herein are described with reference to a “cooling fluid,” it should be understood that such embodiments can generally utilize a thermal regulation fluid that can either be used for heating and cooling; the scope of the present invention includes working in either thermal modality. The system 10 also includes a head covering device or cap 14 and, optionally, a body covering device 16 coupled to the console 12. In one embodiment, the head cap 14 can be removably connected to console 12 by an umbilical 20 having a fluid inlet tube 22 and a fluid outlet tube 24 and the body covering device 16 can be removably connected to console 12 by an umbilical 26 having a fluid inlet tube 28 and a fluid outlet tube 30. In use, the head cap 14 and body covering device 16 can be placed in contact with a patient's head and body, respectively. The console 12 can then circulate the cooling fluid through the head cap 14 and the body covering device 16, via a pump 15, to cause the devices 14, 16 to exchange thermal energy with, and induce hypothermia in, the patient.

The console 12 can further include a temperature sensor 18 that is configured to attach onto an outer surface or within a natural orifice of a patient's body to measure the temperature of the patient during operation of the thermal regulation system 10. For example, in one embodiment, the temperature sensor 18 is an esophageal temperature sensor configured to insert within an esophagus of a patient to measure core body temperature. In another embodiment, the body temperature sensor 18 is a bladder temperature sensor or a tympanic temperature sensor configured to insert within a bladder or ear, respectively, of the patient.

In one embodiment, the temperature of the cooling fluid can be adjusted by the console 12 to control the temperature of the patient's body. For example, the console 12 can include a thermal adjustment device 36, such as a refrigeration mechanism, that can regulate the temperature of the thermal regulation fluid carried by the reservoir 13. During operation, the thermal adjustment device 36 can increase or decrease the temperature of the cooling fluid held in the reservoir 13 in response to signals received from the body temperature sensor 18. The thermally adjusted cooling fluid can then be delivered to the head cap 14 and the body covering device 16 to adjust the patient's body temperature.

In another embodiment, the console 12 can also include a flow rate adjustment mechanism 38 to adjust the flow of thermal regulation fluid from console 12 to the head covering device 14 and the body covering device 16. For example, flow rate adjustment mechanism 38 can be a computerized controller (e.g., a processor and memory) that forms a feedback loop with the body temperature sensor 18 and the pump 15. In response to the signals received from the body temperature sensor 18, the controller 38 can adjust the rate of delivery of cooling fluid by the pump 15 to the head cap 14 and the body covering device 16. During operation, an increase in the rate of delivery of cooling fluid to the head cap 14 and the body covering device 16 can increase the cooling rate in the patient while a decrease in the rate of delivery of cooling fluid can decrease the cooling rate in the patient.

Consoles and/or associated systems (and pieces thereof) can utilize a variety of configurations, and include a variety of features such as including portable units, disposable units, etc. Some such configurations and features are included in U.S. patent application Ser. No. 11/437,413, filed May 19, 2006, entitled “Methods and Apparatus for Thermally Activating a Console of a Thermal Delivery System,” which is hereby incorporated herein by reference in its entirety.

The body covering device 16 can have a variety of configurations. For example, the body covering device 16 can be configured as a neck collar, an axilla pad, or a back pad as described in International Application No. PCT/US2006/012561, entitled “Methods and Apparatus for Thermal Regulation of a Body. In use, the body covering device 16 receives thermal transfer fluid from the console 12 and exchanges thermal energy with the patient's body to induce heating or cooling in the patient (e.g., localized hypothermia). It should be understood that features and functionalities attributed to a head cap as described herein, can also be used with other body covering devices such as neck collars, axilla pads, back pads, etc. For example, the use of two separated thermal fluids that separate the fluid that contacts a body part from the fluid that is circulated through a console or other fluid regulation device can be used in other body

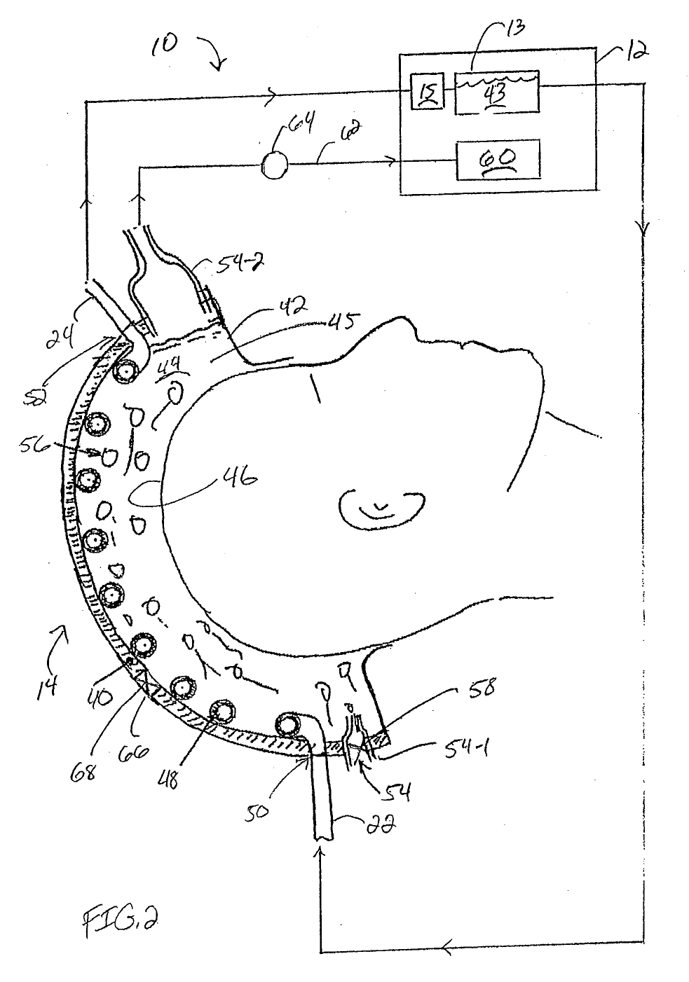

While the head cap 14 can be configured in a variety of ways, FIG. 2 illustrates one embodiment of the head cap 14 of the present invention. A head cap can generally include some type of head-covering for surrounding at least a portion of a subject's head. The head-covering can be embodied in a number of configurations such as being assembled from one or more pieces. The head-covering can be constructed using a variety of materials such as pliable materials or harder materials. In an exemplary embodiment, as depicted in FIG. 2, the head-covering includes a shell 40 formed from a relatively rigid material, such as a polycarbonate material and a sealing member 42 disposed about a periphery of the head cap 14. Of course, the teachings of the present invention can also be applied to other head coverings (e.g., a pliable form fitting cap). The shell 40 and sealing member 42, along with a patient's scalp 46, define a fluid containment space 44 configured to hold a fluid 45, such as saline or Ringer's solution for example. The shell 40 and sealing member 42 are operable to maintain the fluid 45 contained within the fluid containment space 44 in contact with the patient's scalp 46. For example, the sealing member 42 can limit or prevent the fluid 45 from leaking past the rim of the head cap 14. While the sealing member 42 can have a variety of configurations, the sealing member 42 can be configured as an elastomeric band such as described in concurrently filed U.S. Provisional Application entitled “Adjustable Cooling Cap” bearing attorney docket number 104891-43, the contents of which are herein incorporated by reference in their entirety. Other features of body covering devices are described in U.S. Pat. No. 7,052,509 entitled “Method and Device for Rapidly Inducing and Then Maintaining Hypothermia,” issued May 30, 2006, the contents of which are herein incorporated by reference in their entirety.

The head cap 14 also includes a fluid circulation apparatus 48 that can be disposed within the fluid containment space 44 between the shell 40 and the patient's scalp 46. In general, a fluid circulation apparatus can be configured to promote heat transfer between fluid that contact the patient's head and another fluid that can act as a heat transfer medium. For instance, the fluid circulation apparatus can be constructed of materials having a high thermal conductivity, and can be configured to have a high heat transfer coefficient (e.g., having a structure with substantial surface area to promote heat transfer). The fluid circulation apparatus 48, such as a tubular structure disposed within the shell 40, can include an inlet 50 that couples to the fluid inlet tube 22 and pump 15 and an outlet 52 that couples to fluid outlet tube 24. The fluid circulation apparatus 48 is operable to allow circulation of thermal transfer fluid 43 from the reservoir 13 and through the fluid containment space 44 while isolating the thermal transfer fluid 43 from the fluid 45. In general, however, it is desirable to allow, or even promote, heat transfer between the fluids 43, 45, with the console 12 acting to thermally regulate thermal transfer fluid 45, and thus heating or cooling fluid 43. As such, the thermal transfer fluid 43 within the fluid circulation apparatus 48 does not mix with the fluid 45 in fluid containment space 44 or contact the patient's scalp. The console 12 and the reservoir 13, therefore, do not require cleaning after being used with the head cap 14 and the thermal transfer fluid 43 contained by the console 12 can be subsequently used with other head caps 14 for induction of localized hypothermia in other patients. As well, such a design can potentially allow the use of a thermal transfer fluid 43 that may have sufficient or excellent heat transfer properties while not being of a type in which skin contact is desirable.

The closed loop design of the console 12 and the fluid circulation apparatus 48 can help to minimize the amount of thermal transfer fluid 43 which must be added to the console 12 prior to use. As described above, the thermal transfer fluid 43 does not contact the patient's scalp 46 when circulated through the head cap 14 and, as such, cannot be absorbed by the skin or hair of the patient. As a result, because the system 10 does not substantially lose thermal transfer fluid 43 during operation, only a minimal amount of thermal transfer fluid 43 needs to be added to the reservoir 13 prior to use. For example, a volume of thermal transfer fluid in a range of about 1 liter to 2 liters can be added to the reservoir 13 and can be circulated through the fluid circulation apparatus 48 to provide adequate thermal transfer with the fluid 45 within the fluid containment space 44. This amount of thermal transfer fluid 43 minimizes the overall weight of the console 12, thereby allowing the console 12 to be transported to a patient site.

Additionally, the relatively small volume of fluid 43 being circulated between the console 12 and the fluid circulation apparatus 48 reduces the amount of power required by the pump 15 to circulate the fluid 43. For example, a pump 15 having a minimal power requirement, such as a centrifugal pump, can be used as part of the console 12 to circulate the fluid 43. In some embodiments, the thermal transfer fluid 43 does not undergo a phase transition (e.g., converting from a liquid to a gas) when heat transfer takes place with fluid 44. Such embodiments can be advantageous since the need to recompress the fluid 43 after expansion is eliminated, resulting in further potential savings in equipment costs and power requirements.

The head cap 14, in one embodiment, includes a vent 50 that allows air 56 to flow into the fluid containment space 44 to agitate the fluid 45 contained therein. Agitation of the fluid 45 within the space 44 can increase thermal transfer between the fluid circulation apparatus 48, the fluid 45 and the patient's scalp 46 during operation, thereby increasing the rate of induction and depth of hypothermia in the patient.

The vent 54 can have a variety of configurations. In one embodiment, the vent 54 can include a first vent portion 54-1 coupled to a caudal or rear portion of the head cap 14 and a second vent portion 54-2 coupled to a cephalic or front portion of the cap 14. The first vent portion 54-1 can include a valve 58 that allows air 56 to enter the fluid containment space 44 and minimizes or prevents fluid 45 from flowing out from the space 44 through the first vent portion 54-1. The second vent portion 54-2 is in fluid communication with the fluid containment space 44 and couples to a pump 60, such as an air pump, via connector 62.

In use, the pump 60 can create a vacuum within the fluid containment space 44 thereby causing air 56 to enter the space 44 via the first vent portion 54-1. With the first vent portion 54-1 coupled to the rear portion of the head cap 14 and the second vent portion 54-2 coupled to the front portion of the cap 14, as the air 56 is introduced into the fluid containment space 44 through the first vent portion 54-1, the air or air bubbles 56 float toward the front of the cap 14 to the second vent portion 54-2 for removal from the cap 14 by the pump 60. The relative positioning of the first vent portion 54-1 and the second vent portion 54-2, therefore, minimizes the creation of air pockets within the fluid containment space 44 that can decrease the cooling efficiency or thermal transfer between the patient's scalp and the fluid 45.

As the pump 60 removes air 56 from the fluid containment space 44, in certain cases, the pump 60 can remove a portion of the fluid 45 contained therein. In order to limit the “contaminated” fluid 45 from entering the pump 60, in one embodiment, the connector 62 includes a fluid trap 64 disposed between the second vent portion 54-2 and the pump 60. the fluid trap 64 can substantially remove fluid 45 from a fluid/air mixture suctioned from the fluid containment space 44 by the pump 60. for example, the fluid trap 64 can be a hydrophilic filter that absorbs the fluid 45 and allows passage of air 56 to the pump 60.

As indicated above, the head cap 14 is configured to contain fluid 45 within the fluid containment space 44. The fluid 45 can be introduced into the fluid containment space 44 in a variety of ways. For example, once the head cap 14 has been placed over the patient's head, fluid 45 can be delivered into the space 44 through the vent 54. In another example, the head cap 14 can include one or more fluid ports 66 that allow the fluid 45 to be introduced and maintained within the fluid containment space 44. The port 66 can include a valve 68 that maintains the fluid 45 within the space 44 during use. In one embodiment, the fluid port 66 can also be used as a drain to allow fluid to be removed from the fluid containment space 44.

During operation, the system 10 can induce localized hypothermia in a patient. For example, the head cap 14 can be placed on the head of the patient and fluid 45 introduced within the fluid containment space 44 between the shell 40 and the patient's scalp 46. The pump 15 of the console 12 can then be operated to deliver a cooling fluid 43 from the reservoir 13 to the inlet 50 of the fluid circulation apparatus 48. As the cooling fluid 43 flows from the inlet 50 to the outlet 52, a thermal exchange can occur between the fluid circulation apparatus 48 and the fluid 45 to reduce the temperature of the fluid 45. The pump 60 of the console can also be operated to introduce air bubbles 56 within the fluid containment space 44 to agitate the reduced temperature fluid 45 and enhance thermal transfer between the fluid 45 and the patient's scalp 46. Over time, by exposing the patient to the reduced temperature fluid 45, the system 10 can induce localized hypothermia within the patient's head.

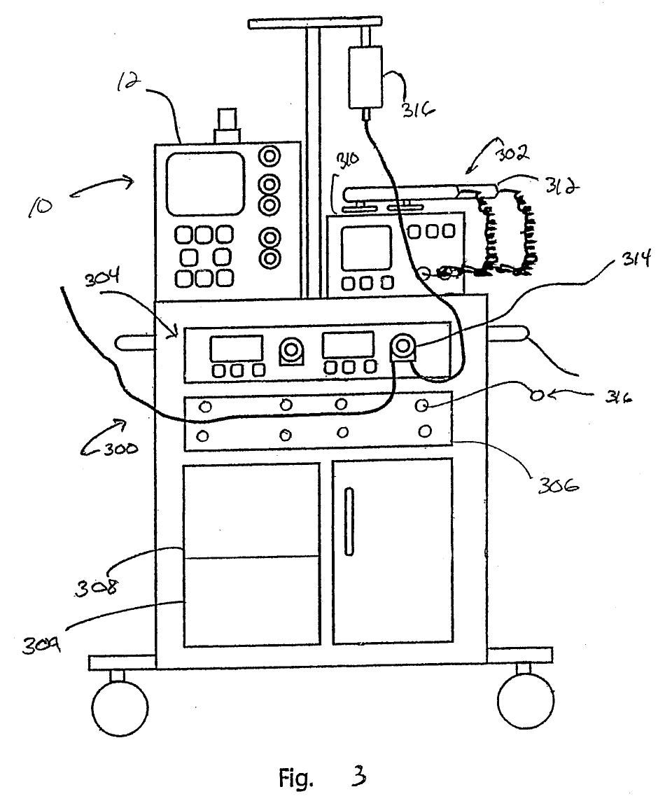

In certain cases, a patient may need to undergo a resuscitation procedure in conjunction with hypothermia therapy. FIG. 3 illustrates an embodiment of the thermal regulation system 10 forming part of a resuscitation system 300 various mechanisms necessary to or used in a resuscitation process. For example, the resuscitation system 300 can include a defibrillation apparatus 302, a fluid treatment apparatus 304, a physiologic monitoring apparatus 306, a ventilator 308, and a chest compression apparatus 309.

The defibrillation apparatus 302 can include a defibrillator 310 and defibrillator electrodes 312. After applying the defibrillation electrodes 312 to a patient and activating the defibrillator 310, an electrical current is provided to the patient's heart to restore a normal rhythm thereto.

The fluid treatment apparatus 304 can include a fluid infusion pump 314 that provides metered infusion of fluids into the patient. The pump 314 can deliver the fluids, such as a Ringer's solution, from a fluid bag 316 to the patient to maintain a hydration level of the patient. In another arrangement the pump 314 can deliver a fluid medicament from the fluid bag 316 to the patient to aid in patient resuscitation.

The physiological monitor 306 and sensor 316 can detect a physiologic state of a patient and can adjust delivery of thermal exchange fluid 43 from the console 12 to the head or body covering devices 14, 16 to adjust or maintain the patient's body temperature based upon the detected physiologic state. For example, the physiological monitor 180 can be an electrocardiogram (ECG) sensor, an electroencephalogram (EEG) sensor, a heart monitoring sensor, a temperature sensor, or a pulse oximetry sensor.

The ventilator 308 can couple to a patient airway and provide oxygen and other gasses to the patient during a resuscitation procedure. The chest compression apparatus 309 can couple to the chest of the patient and can operate in conjunction with the ventilator to cyclically compress the patient's chest and aid in the resuscitation of the patient.

In one embodiment, the thermal regulation system 10 can be used in conjunction with neurological monitoring equipment. For example, the thermal regulation system 10 can be used in conjunction with an intracranial pressure monitoring device. In use, the intracranial pressure monitoring device can measure, for example, a pressure of the cerebrospinal fluid within a patient's brain ventricle. Based upon the pressure measured by the pressure monitoring device, the thermal regulation device 10 can adjust the temperature of the fluid within the ventricle by adjusting the temperature of the thermal regulation fluid 43 delivered to the head cap 14 or body covering device 16 or by adjusting a rate of delivery of the thermal regulation fluid 43 to the head cap 14 or body covering device 16.

One skilled in the art will appreciate further features and advantages of the invention based on the above-described embodiments. Accordingly, the invention is not to be limited by what has been particularly shown and described, except as indicated by the appended claims. Indeed, it is understood any feature of any embodiment can be combined with one or more features of any other embodiment, when compatible, to create other embodiments within the scope of the present invention. All publications and references cited herein are expressly incorporated by reference in their entirety.

What is claimed is:

1. A device for heating or cooling a patient's head, comprising:
a head-covering adapted to at least partially surround the patient's head, the head-covering defining at least a portion of a fluid containment space into which a first thermal transfer fluid can be introduced to contact the patient's head; and
a fluid circulation apparatus disposed at least partially within the fluid containment space, the fluid circulation apparatus adapted to allow circulation of a second thermal transfer fluid through the fluid circulation apparatus while maintaining separation between the second thermal transfer fluid and the first thermal transfer fluid.

2. The device of claim 1, wherein the fluid circulation apparatus is adapted to promote heat transfer between the first thermal transfer fluid and the second thermal transfer fluid.

3. The device of claim 2, wherein the fluid circulation apparatus comprises a tubular structure with an outer surface at least partially disposed within the fluid containment space.

4. The device of claim 1, wherein the head-covering comprises a shell.

5. The device claim 1, wherein the fluid containment space is located between a surface of the head-covering and the patient's head when the head-covering is placed on the patient's head.

6. The device of claim 1, wherein the head-covering comprises a vent in fluid communication with the fluid containment space.

7. The device of claim 6 wherein the vent comprises an air inlet and an air outlet.

8. The device of claim 7 wherein the air inlet is disposed at a caudal portion of the head-covering.

9. The device of claim 7 wherein the air outlet is disposed at a cephalic portion of the head-covering.

10. The device of claim 1, wherein the device is adapted to cool the patient's head.

11. The device of claim 1, further comprising:
at least one fluid port in communication with the fluid containment space for introducing or removing fluid from the fluid containment space.

12. A method of heating or cooling a patient's head, comprising:
contacting the patient's head with a first thermal transfer fluid;
transferring heat between the first thermal transfer fluid and a second thermal transfer fluid, the second thermal transfer fluid being separated from the first thermal transfer fluid and being capable of thermal regulation to control heating or cooling of the patient's head.

13. The method of claim 12, further comprising:
providing a cap to contain the first thermal transfer fluid between the cap and the patient's head when the cap is worn by a patient.

14. The method of claim 13, further comprising:
placing the cap on the patient's head and subsequently inserting the first transfer fluid into the cap.

15. The method of claim 12, wherein the method is used to induce localized hypothermia in a patient.

16. The method of claim 12, wherein the step of transferring heat comprises circulating the second thermal transfer fluid in a closed loop circulation apparatus to thermally regulate the second thermal transfer fluid.
